(19) United States
(12) Patent Application Publication
(10) Pub. No.: US 2010/0322979 A1
(43) Pub. Date: Dec. 23, 2010

(54) Peptidomimetic Resorbable Peptide-Polymer Hybrid Polyester Nanoparticles

(75) Inventor: Esmaiel Jabbari, Columbia, SC (US)

Correspondence Address: DORITY & MANNING, P.A., POST OFFICE BOX 1449, GREENVILLE, SC, 29602-1449, US

(73) Assignee: UNIVERSITY OF SOUTH CAROLINA, Columbia, SC (US)

(21) Appl. No.: 12/820,683

(22) Filed: Jun. 22, 2010

Related U.S. Application Data
(60) Provisional application No. 61/269,224, filed on Jun. 22, 2009.

(51) Int. Cl. A61K 9/14; A61P 35/00; A61K 31/337; B32B 1/00

(52) U.S. Cl. 424/400; 514/449; 428/402; 424/497; 977/773

(57) ABSTRACT

In accordance with certain embodiments of the present disclosure, a self-assembling biodegradable nanoparticle is provided. The nanoparticle includes a degradable synthetic polymer chain, a sequence of non-polar amino acids, and a sequence of ionic amino acids. The nanoparticle has a diameter of from about 50 nm to about 150 nm.

### CROSS-REFERENCE TO RELATED APPLICATION

The present application is based on and claims priority to U.S. Provisional Application 61/269,224 having a filing date of Jun. 22, 2009, which is incorporated by reference herein.

### BACKGROUND

Drugs used in chemotherapy are highly toxic; that is they destroy the cancerous tissue as well as other normal tissues in cancer patients. This causes intense side effects such as fever, sweating, pain, fatigue, gastrointestinal complications, cognitive disorders, and death. Techniques that can selectively target the anticancer drug to the tumor environment while bypassing normal healthy tissues has the potential to eliminate side effects, thus reducing patient suffering and recovery time, and increase survival rate of cancer patients. The tumor environment differs significantly from that of normal tissues, and by taking advantages of these differences, it is possible to selectively target toxic anticancer drugs to the cancerous tissue while leaving normal tissues unharmed. Tumor blood vessels are abnormal compared with normal vessels, resulting in higher permeability of tumor tissue. This means that particles with less than 150 but greater than 50 nanometer size are preferentially taken up by tumor, compared to normal tissues. Therefore, if the anticancer drug is attached to particles with size in the range of 50-150 nanometers and administrated systemically, a larger fraction of the drug is taken up by tumor tissue. Second, most tumors lack lymph vessels and higher interstitial fluid pressure than normal tissues, so interstitial fluid and soluble macromolecules are inefficiently removed. Therefore, particles have to degrade to molecular weights <50 kDa to avoid their accumulation in the interstitium (EPR effect) which retards their additional uptake from blood vessels to tumor interstitial space. Third, the particles should provide a sustained dose of the chemotherapeutic agent in the tumor environment throughout the chemotherapy schedule to improve efficiency. Fourth, particles that are modified with ligands that preferentially interact with tumor-associated cell surface receptors improve selectivity and increase residence time of the particles in the tumor tissue. Ligand conjugated particles are very attractive as a mechanism for cell-selective tumor drug delivery, since this process has high transport capacity as well as ligand dependent cell specificity.

In view of the above, a need exists for particles with size in the range of 50-150 nanometers which can effectively retain and release chemotherapeutic agents. In addition, it is desired that such nano-carriers degrade by biochemical pathways in the organism, to prevent accumulation in the interstitium, and prevent the loss of bioactivity of the agent to be delivered.

### SUMMARY

In accordance with certain embodiments of the present disclosure, a self-assembling biodegradable nanoparticle is provided. The nanoparticle includes a degradable synthetic polymer chain, a sequence of non-polar amino acids, and a sequence of ionic amino acids. The nanoparticle has a diameter of from about 50 nm to about 150 nm.

In still other embodiments of the present disclosure, a self-assembling biodegradable nanoparticle is provided. The nanoparticle includes Cys-Val-Val-Val-Val-Val-Val-Lys-Lys conjugated with a synthetic polymer and has a diameter of from about 50 nm to about 150 nm. The nanoparticle also includes a therapeutic agent wherein the nanoparticle is configured to have a generally linear profile of release for the therapeutic agent.

Other features and aspects of the present disclosure are discussed in greater detail below.

### BRIEF DESCRIPTION OF THE DRAWINGS

A full and enabling disclosure, including the best mode thereof, directed to one of ordinary skill in the art, is set forth more particularly in the remainder of the specification, which makes reference to the appended figures in which:

FIG. 1 illustrates images of PLGF-PLEOF (a) and CV6K2-PLGF (b) NPs; size distribution of PLGF-PLEOF, CV6K2, and CV6K2-PLGF NPs (c).

FIG. 2 illustrates (a) Mass loss of CV6K2-PLAF and PLAF-PLEOF NPs; (b) Release kinetics of the model Paclitaxel drug from CV6K2, CV6K2-PLAF, and PLAF-PLEOF NPs with incubation time.

FIG. 3 illustrates fluorescent images of cell nuclei (a) cytoskeleton (b) and FITC-loaded NPs (c) for HCT116 tumor cells incubated with FITC-dextran loaded NPs; (d) is cell viability of HCT116 cells incubated with NPs, free Paclitaxel, and Paclitaxel encapsulated in NPs; (e) is cell viability of BMS cells incubated with 75 and 150 mg/ml CV6K2-PLGF NPs with time.

FIG. 4 illustrates (a) whole animal near-infrared image of Apc^{Min/+} mouse with intestinal tumor injected with dye-loaded PLAF NPs; (b) and (c) are the near infrared images of the intestine of a normal (b) and Apc^{Min/+} mice 4 h after injection of dye-loadedPLAF NPs.

FIG. 5 illustrates fluorescent image of MCF-7 (a) and U87MG (b) cells. The dots in (b) are the FITC-stained peptide grafted NPs.

FIG. 6 illustrates (a) ESI-MS of CV6K2 peptide and (b) size distribution of peptide-conjugated NPs.

FIG. 7 illustrates (a) XPS of the conjugated NPs and (b) TEM of the CV6K2-PLAF NPs.

FIG. 8 illustrates the release profile of Paclitaxel from PLAF-CV6K2 and PLGF-CV6K2.

FIG. 9 illustrates cell viability after incubation with peptide NPs.

FIG. 10 illustrates mice body weight (a) and tumor volume (b) with time after receiving one of the treatments; (c) NIR image of Apc^{Min/+} mouse with intestinal tumor injected with dye+PLAA-EO NPs (inset is image of the isolated intestine). In (a) one star means s.d. (p=0.05) between the test group and PBS, PLAA-EO, PLAA-CV6K2, and Dox+PLAA-EO NPs (the 4-groups). Two stars in (a) means s.d. between the test group and Dox+PLAA-CV6K2 NPs. One star in (b) means s.d. between the test group and the 4-groups.

### DETAILED DESCRIPTION

Reference now will be made in detail to various embodiments of the disclosure, one or more examples of which are set forth below. Each example is provided by way of explanation of the disclosure, not limitation of the disclosure. In fact, it will be apparent to those skilled in the art that various modifications and variations can be made in the present disclosure without departing from the scope or spirit of the disclosure. For instance, features illustrated or described as part of one embodiment, can be used on another embodiment to yield a still further embodiment. Thus, it is intended that the present disclosure covers such modifications and variations as come within the scope of the appended claims and their equivalents.

The present disclosure is directed to synthesis of peptidomimetic functionalized self-assembled poly(lactide-co-glycolide) nanoparticles (NPs) with narrow size distribution, constant degradation rate with sustained release of the chemotherapeutic agent that can be conjugated with ligands that preferentially interact with tumor cells. Although the ideal application of these peptidomimetic nanoparticles is in tumor delivery, other practical and important applications include as adjuvant in vaccination, as sustained targeted release system in drug and protein delivery, gene delivery, growth and differentiation factor delivery in regenerative medicine, fluorescent biological labeling, detection of proteins and pathogens and probing the DNA structure, separation and purification of biological molecules and cells, and in imaging as contrast agent.

NPs are being considered for their applications in targeted drug delivery to tumor tissue. Tumor tissue has increased permeability (EPR effect), which enables particles of less than 200 nm in size to be selectively taken up by tumor vasculature. Surface-modified NPs have the potential to increase the effectiveness of targeted delivery by means of introducing specific ligands that can bind with high specificity to receptors on the cell surface. It is desired that these nano-carriers degrade by biochemical pathways in the organism, to prevent accumulation in the interstitium, and prevent the loss of bioactivity of the agent to be delivered. The present disclosure describes synthesis of biodegradable NPs from poly(actide-co-glycolide fumarate) (PLGF) macromer for targeted delivery of bioactive agents. It has been demonstrated that the peptide sequence Cys-Val-Val-Val-Val-Val-Val-Lys-Lys (CV6K2) self assembles in aqueous solution into NPs. The addition of CV6K2 sequence to PLGF macromer facilitates assembly of the macromer to NPs. In this regard, the present disclosure describes synthesis of PLAF and PLGF macromers conjugated to the CV6K2 sequence, evaluates their self-assembly properties, and characterizes them in terms of their morphology, size, degradation properties, release characteristics, and cell uptake.

However, other suitable structures are contemplated by the present disclosure. In this regard, the NP of the present disclosure includes a macromer. The macromer includes a degradable polymer chain. Suitable degradable polymers include poly(lactide) and poly(glycolide) and their copolymers (PLGA), poly(caprolactone) (PCL) and its copolymers with PLGA, polypropylene fumarate) and its copolymers with PLGA and PCL, polyhydroxyalkanoate (PHA), copolymers of PLGA with poly(ethylene glycol) (PEG), poly(anhydrides), polydioxanone, poly(trimethylene carbonate), poly(ester amides), poly(ortho esters), poly(amino acids), polyphosphazenes, and polyphosphoesters, and combinations thereof. Other suitable degradable polymers are known in the art and are described in Nair L. S., Laurencin C. T., Biodegradable polymers as biomaterials (2007) Progress in Polymer Science (Oxford), 32 (8-9), pp. 762-798, incorporated by reference herein. In addition, the degradable polymer can be joined with a non-degradable polymer.

The macromer is joined with a sequence of non-polar amino acids and a sequence of ionic amino acids. Suitable non-polar amino acids include valine, glycine, alanine, leucine, isoleucine, methionine, proline, phenylalanine, tryptophan, and combinations thereof. Suitable ionic amino acids include lysine, arginine, histidine, aspartic acid, glutamic acid, and combinations thereof.

The nanoparticle of the present disclosure can have a diameter of from about 50 nm to about 150 nm and can carry one or more suitable bioactive agents, including enzymes, organic catalysts, ribozymes, organometallics, proteins, glycoproteins, peptides, polyamino acids, antibodies, nucleic acids, steroidal molecules, antibiotics, antimycotics, cytokines, growth factors, carbohydrates, oleophobics, lipids, extracellular matrix and/or its individual components, pharmaceuticals, therapeutics, and combinations thereof.

The present disclosure can be better understood with reference to the following examples.

### EXAMPLES

### Example 1

### Macromer Synthesis and Production of Nanoparticles (NPs)

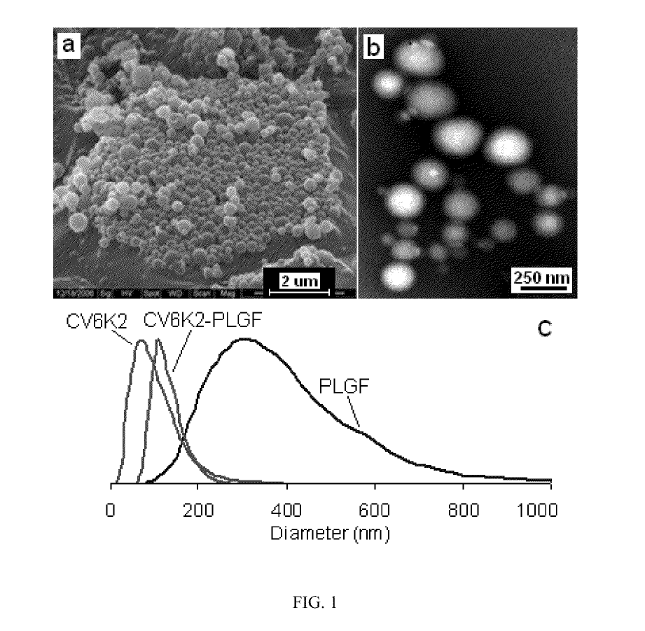

Fumarate functionalized poly(lactide-co-glycolide) (PLGF) NPs are conjugated with CV6K2 peptide to stabilize the NPs through specific interactions between the amino acid side chains. Low molecular weight poly(lactide-co-glycolide) (LMW PLGA) are synthesized by ring opening polymerization of a mixture of lactide and glycolide monomers. PLGF was synthesized by polymerization of ULMW PLGA with fumaryl chloride (FuCl). The fumarate groups in PLGF macromer provide functionality for covalent attachment of ligands which bind with high specificity to receptors on tumor-associated cells, like the αᵥβ₃ integrin receptor. ULMW PLGA with number-average molecular weight in the range 1-3 kDa was used in the synthesis of PLGF for highest density of unsaturated fumarate groups for ligand conjugation. Number average molecular weights of PLGF and PLEOF were in the range of 6-15 kDa and 10-20 kDa, respectively. The CV6K2 peptide, synthesized manually in the solid-phase, was conjugated to PLGF macromer by the reaction between the sulfhydryl group of cystine with fumarate group of PLGF. PLGF-PLEOF and CV6K2-PLGF NPs were produced by dialysis of the macromers in dimethylsulfoxide (DMSO)/N,N-dimethylformamide (DMF) against water. The morphology and size distribution of the NPs were determined by SEM and dynamic light scattering, respectively. Electron micrographs in FIGS. 1(a) and 1(b) show the images of PLGF-PLEOF and CV6K2-PLGF NPs, respectively. These images demonstrate that conjugation of CV6K2 to PLGF results in NPs with significantly smaller size ranging from 50-150 nm. Average size of the NPs can be varied from 500 to 15 nm by adjusting the PLGF molecular weight and number of peptides per macromer to for selective targeting to tumor vasculature.

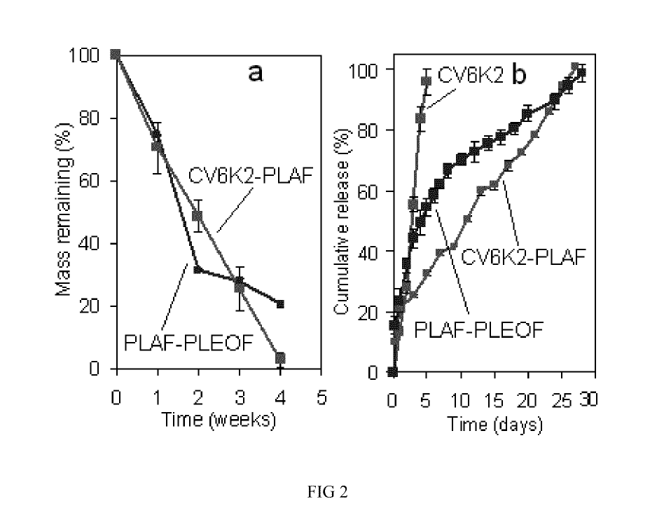

Degradation and release characteristics of NPs. PLGF macromer with 100:0 lactide to glycolide ratio (PLAF) was synthesized by condensation polymerization. CV6K2 peptide was conjugated to PLAF to produce CV6K2-PLAF macromer. PLGF-PLEOF and CV6K2-PLGF NPs were produced by dialysis of the macromers in dimethylsulfoxide/dimethylformamide (DMSO/DMF) against water. The degradation of the NPs was measured in phosphate buffer saline (PBS) at 37° C. and the results are shown in FIG. 2(a). Degradation of PLGF-PLEOF NPs was non-linear with incubation. Unusual constant linear mass loss in 4 weeks was observed for CV6K2-PLAF NPs, which can be explained by erosion of the PLGF macromers with time. To measure release kinetics, Paclitaxel was used as the surrogate molecule. Paclitaxel loaded NPs were prepared by dialysis. After dialysis, the NPs were dissolved in DMSO and 3 ml of acetonitrile-water mixture (50:50 v/v) was added. After 2 h, the suspension was centrifuged and the amount of Paclitaxel in the supernatant was measured by isocratic reverse-phase HPLC with a photodiode array detector at the wavelength of 227 nm. The encapsulation efficiency ranged from 70 to 56% and decreased with increasing Paclitaxel concentration. The release kinetics of Paclitaxel from NPs was measured in-vitro in PBS (pH 7.4) and the results are shown in FIG. 2(b). The release kinetics followed the degradation of the NPs, shown in FIG. 2(a). The release kinetics of Paclitaxel from PLAF-PLEOF NPs was non-linear (FIG. 2b) with incubation time while that of CV6K2-PLAF NPS was linear (FIG. 2b). Furthermore, CV6K2-PLGF NPs (FIG. 2b) released their content in 4 weeks while CV6K2 NPs (FIG. 2b) released in 3 days. These results demonstrate that peptidomimetic NPs, with relatively narrow size distribution can release the drug at a constant rate while degrading to prevent NPs accumulation.

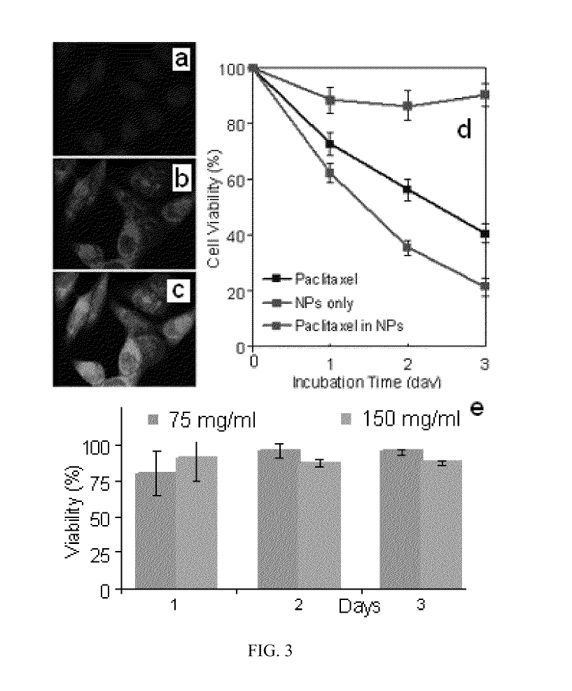

NPs uptake by tumor cells. HCT116 cancer cell line (America Type Culture Collection (ATCC)) were cultured in McCoy's Medium supplemented with 10% FBS and harvested with trypsin/EDTA. After reaching 70% confluency, medium was replaced with FITC-dextran loaded NPs suspension in basal media (250 mg/ml, pH 7.4). After incubation for 2 h, the free NPs were removed by washing with PBS, fixed, and cells were stained with phalloidin (for cytoskeleton) and DAPI (for cell nuclei) and imaged by confocal laser scanning microscopy. The fluorescent images of the cell nuclei, cytoskeleton, and FITC-loaded NPs are shown in FIGS. 3(a), 3(b), and 3(c), respectively. These images demonstrate that the FITC-loaded NPs are internalized by tumor cells (green fluorescence of the NPs coincides with red fluorescence of the cytoskeleton). The particle uptake efficiency was both time and concentration dependent. Similar results were obtained when FITC-loaded NPs were incubated with WM115, HT29, DLD1, and 4T1 tumor cell lines.

Tumor cell toxicity was determined by incubating Paclitaxel drug, encapsulated in NPs, with HCT116 tumor cell line, seeded at a density of 5×10⁴ cells/cm² and cultured in basal media (40 μg/ml Paclitaxel) for 24, 48, and 72 h. At each time point, suspension was removed, cells were washed with PBS, and viability was assessed by the MTT assay. FIG. 3(d) compares cell viability of HCT116 cells (expressed as the percentage of the values obtained from cells in the presence of drug compared to those in drug-free samples) incubated with 40 μg/ml Paclitaxel with that incubated with the same concentration encapsulated in PLGF NPs. The line shows the cell viability of the NPs without Paclitaxel. The viability of HCT116 cells incubated with empty NPs was >90% demonstrating that NPs pose little toxicity to cells. Cell viability of Paclitaxel in solution after 3 days was 40% while that encapsulated in NPs was 28%. Cell viability of the drug-free NPs was >90% demonstrating that NPs posed little toxicity to cells. Cell viability of Paclitaxel in solution after 3 days was 40% while that encapsulated in NPs was 28%. Toxicity of CV6K2-PLGF NPs to normal cells was assessed with bone marrow stromal (BMS) cells isolated from the bone marrow of Wistar rats. BMS cells were seeded in 24 well plates in primary media at a density of 4×10⁴ cells/cm², incubated for 24 h for cell attachment. After cell attachment, the media was replaced with media containing 75 or 150 mg/ml of CV6K2-PLGF NPs and incubated for 1, 2, and 3 days. At each time point, the cells were washed with PBS to remove the free NPs, and the fraction of viable cells was measured by the MTT assay. FIG. 3(e) shows fraction of live cells was >80 for all time points. This result demonstrates that CV6K2-PLGF NPs do not have significant cytotoxicity toward normal cells.

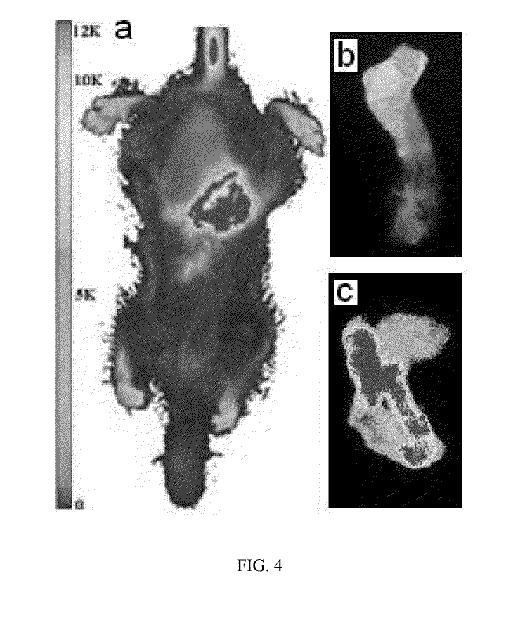

Determination of NPs biodistribution by live animal imaging. Near-infrared imaging was used to determine in-vivo distribution of NPs. The near-infrared dye IRDye 800RS Carboxylate (LI-COR Biosciences), with peak absorption at 786 nm, was loaded in PLGF NPs. 500 μA of the NPs suspension was injected in the tail vein of the male Apc^{Min/+} mice with intestinal tumor (6 months old; Jackson Laboratories). The mice were anesthetized with 4.5% isoflurane in an oxygen carrier gas and transferred to the MousePOD Adapter scanning surface of an Odyssey Infrared Imaging System (model 9201-3; LI-COR Biosciences). The animals were scanned tail to head in two infrared channels simultaneously (700 and 800 nm) where one channel (700 nm) was used to normalize intensities. Near-infrared image (displayed in pseudo colors) of the Apc^{Min/+} mouse 4 h after injection with PLGF NPs is shown in FIG. 4(a). The infrared intensity in the intestinal region was at least 100 times higher than the other regions (confirmed by sacrificing the animal, removing and directly imaging the intestinal tissue). Since the animal was injected in the tail vein, relatively high intensity was also observed in the tail. After whole animal scanning, the animal was sacrificed, the intestinal tissue was isolated, and the relative amount of NPs in each organ was qualitatively measured by infrared imaging. FIGS. 4(b) and 4(c) compare the near infrared images of the intestine isolated from a normal mouse (b) with that isolated from an Apc^{Min/+} mouse 4 h after injection of the dye-loaded PLAF NPs. FIGS. 4(b) and 4(c) clearly demonstrate that there is selective uptake of the NPs by the tumor tissue due to their size.

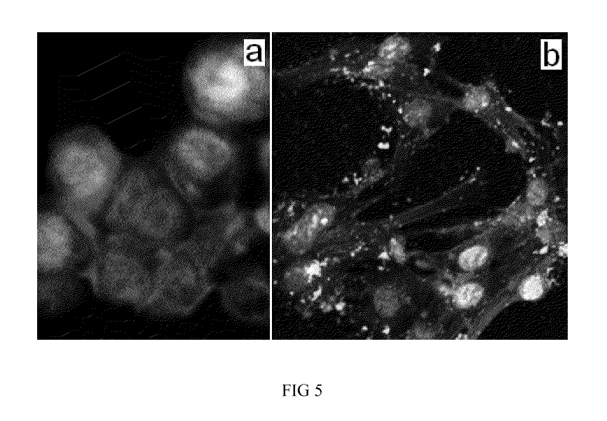

Targeting to tumor endothelial cells by grafting cyclic c(-RGDfC-) peptide to NPs: The linear D-Phe-Cys-Arg-Gly-Asp peptide was synthesized manually using Fmoc chemistry. After peptide chain elongation, the linear peptide was cyclized directly on the peptidyl resin by coupling the carboxylate group on aspartic acid to the amine group on phenylalanine in the peptide sequence. The cyclized peptide was side-chain deprotected, cleaved from the resin, precipitated in cold ether, and purified by preparative high-performance liquid chromatography (HPLC). The cyclic peptide was characterized by Electro Spray Ionization (ESI) spectrometry For grafting, c(-GRGfC-) peptide was incubated with NPs at ambient conditions for 10 h. After grafting, the NPs were purified by dialysis against water and incubated with MCF-7 and U87MG tumor cells. After incubation, unattached NPs were removed by washing, cells were fixed, and stained with phalloidin (for cytoskeleton) and DAPI (for cell nuclei) and imaged with confocal microscopy. FIGS. 5(a) and 5(b) show the image of MCF-7 and U87MG cells, respectively. The bright dots in FIG. 5(b) and their absence in FIG. 5(a) are the FITC-stained c(-GRGfC-) peptide grafted NPs that are attached/internalized by U87MG cells that have high expression of αᵥβ₃ integrin receptor. The images in FIG. 5 demonstrate that the c(-GRGfC-) grafted NPs bind to tumor cells which express αvβ3 integrin receptor.

These results demonstrate that CV6K2-PLGF peptidomimetic NPs have very narrow size distribution, have linear degradation kinetics, and release drugs at a constant rate with time. In addition, results demonstrate that the CV6K2-PLGF NPs can be conjugated with bioactive peptides to design cell-responsive NPs for tumor targeting and other biological applications. This invention can be used to synthesize NPs with other polyesters like polycaprolactone, with unsaturated groups other than fumarate like acrylate and methacrylate, and with other peptide sequences that self-assemble to form NPs. The example provided here is one of many embodiments of this invention. Applications of this invention include targeted delivery of chemotherapeutic agents in cancer therapy, as adjuvant in vaccination, as sustained resorbable release systems in drug, protein, and gene delivery, growth and differentiation factor delivery in regenerative medicine, fluorescent biological labeling, detection of proteins and pathogens and probing the DNA structure, separation and purification of biological molecules and cells, and in imaging as contrast agent.

### Example 2

### PLAF and PLGF Synthesis

The low molecular weight poly(lactic acid) (PLA) and poly(lactide-co-fumarate) (PLGA) were synthesized by ring-opening polymerization of lactide and/or glycolide monomers with diethylene glycol as the initiator and tin hexanoate as the polymerization catalyst. The molar ratio of DEG to TOC was 25:1. Next, PLAF or PLGF was synthesized by condensation polymerization of PLA or PLGA, respectively, with fumaryl chloride (FuCl). The amphiphilic poly(lactide-co-ethylene oxide-fumarate) (PLEOF) macromer was synthesized by condensation polymerization of ultra-low-molecular weight poly(L-lactide) (ULMW-PLA) and poly(ethylene glycol) (PEG) with fumaryl chloride (FuCl) and triethylamine (TEA) as the catalyst as described. Triethylamine (TEA) was used as the acid scavenger. For PLEOF, The molar ratio of FuCl:PEG and TEA:PEG was 0.9:1.0 and 1.8:1.0, respectively. The synthesized macromers were characterized by ¹H-NMR and gel permeation chromatography (GPC).

Self-assembly peptide synthesis and conjugation to macromers: The peptide sequence CVVVVVVKK (CV6K2) was synthesized manually on 200 mg of Rink Amide NovaGel resin (0.62 mmol/g). The Fmoc-protected amino acid derivative (1 equiv) and hydroxybenzotriazole (HOBt; 2 equiv) were dissolved in dry N,N-dimethylformamide (DMF; 3 mL), and N,N-diisopropylcarbodiimide (DIC; 1.1 equiv) was added to the mixture. Next, 0.2 mL of 0.05 M N,N-dimethylaminopyridine (DMAP) was added, and the mixture was shaken for 4-6 h at 30° C. in an orbital shaker. A small amount of resin was removed and tested for the presence of unreacted amines using the Kaiser reagent; the coupling reaction was repeated until a negative result was obtained. Then, the resin was washed thoroughly with DMF, treated with 20% piperidine in DMF for Fmoc deprotection, and washed with DMF. The subsequent amino acids were coupled using the same method. After coupling and deprotecting the last amino acid of the sequence, the resin was treated with 95% trifluoroacetic acid (TFA)/2.5% triisopropylsilane (TIPS)/2.5% water for 2 h to cleave the peptide from the resin. The peptide was precipitated in cold ether and dried. The dried product was characterized by mass spectrometry. The macromer was then reacted with the peptide, making a thioether bond, by reacting the fumaryl group of the macromer with the sulfhydryl group of the cysteine. A mixture of peptide and macromer (2:1 peptide:macromer molar ratio) was dissolved in a 1:5 solution of DMF:water and placed in an orbital shaker at 20° C. for at least 12 h. Next, the solution was dialyzed against deionized (DI) water and freeze-dried to obtain PLAF-CV6K2 and PLGF-CV6K2. The macromers were characterized by GPC and mass spectrometry.

Nanoparticle (NP) self-assembly and characterization: PLAF-CV6K2 or PLGF-CV6K2 macromers were dissolved in a solution of 1 mL DMF and 8 mL dimethysulfoxide (DMSO). The solution was loaded in the dialysis tube (molecular cutoff: 3.5 kDa) and dialyzed against phosphate-buffer saline (PBS). The solution was dialyzed for 24 h with change of dialysis buffer every 2-4 h until DMSO and DMF were completely removed. The resulting NP solution was used for experimentation. PLAF and PLGF NPs were synthesized similarly, with the addition of 10% wt PLEOF macromer. The morphology and size distribution of the NPs was examined by TEM. The sample was placed on the TEM grid and allowed to dry, stained with uranyl acetate, and observed at an accelerating voltage of 200 keV. The size distribution of NPs was measured by dynamic light scattering (DLS). The scattered light intensity was inverted to size distribution by inverse Laplace transform. X-ray photoelectron spectroscopy (XPS) measurements were performed on the NPs to determine the elemental composition of their external surface. Degradation of the NPs was followed by measuring their particle size and mass loss as a function of incubation time. 50 mg NPs were suspended in 1 mL PBS and the suspensions were incubated at 37° C. until complete degradation. At each time point, NPs size was measured with DLS. Suspensions were centrifuged at 15000 rpm and the supernatants collected for freeze-drying. The fraction of mass remaining was determined by dividing the dried mass in the supernatant at time t by the initial mass at time zero.

Release profiles of Paclitaxel from CV6K2 NPs: NPs (6% Paclitaxel by weight of the PLAF or PLGF macromer) were prepared by dialysis. After dialysis for 24 h to self-assemble the macromers and to remove the unencapsulated Paclitaxel, encapsulation efficiency was determined by HPLC. The release kinetics of Paclitaxel from the NPs was measured in-vitro in PBS (pH 7.4) for up to 28 days. At each time point, the suspension was centrifuged at 15,000 rpm for 10 min, and the supernatant was removed and transferred into microvials for HPLC analysis. The precipitate was re-suspended in 10 mL fresh PBS. The suspension was maintained in PBS at 37° C. with orbital shaking until the next time point.

Cell uptake and viability: To measure NPs uptake, bone marrow, stromal (BMS) cells were seeded at a density of 5×10⁴ cells/cm² per well in 96-well plates and incubated for 2 hours. Afterwards, the basal media was replaced with media supplemented with the NPs with concentrations of 0.17 mg/mL (1×), 0.68 mg/mL (4×) and 1.36 mg/mL (8×) NP for 24, 48 and 72 hours. At each time point, the media was collected and lyophilized, and the mass of NPs in solution was determined. For cell viability, cells were exposed to NPs for 2 hours, and then fresh media was used. Cell viability was determined by dividing the number of cells by the control well (no NPs).

The calculated molecular weight of the peptide was 1013 Da. In the ESI-MS spectrum, mass numbers (m/z) 1014 and 1036 corresponded to the monovalent hydrogen cation [(M+H)⁺] and monovalent sodium cation [(M+Na)⁺] of the peptide, respectively (FIG. 1a). M̅ₙ̅, M̅_̅{̅w̅}̅, and polidispersity index (PDI) of the PLAF were 5294, 10574, and 1.99, respectively; for the PLGF, the values were 6613, 11641 and 1.86, respectively. M̅ₙ̅, M̅_̅{̅w̅}̅, and PDI of the PLAF-CV6K2 were 5808, 11249, and 1.93, respectively; for the PLGF-CV6K2, the values were 7458, 12596 and 1.69, respectively. The increase of molecular weight indicated that there was an average of at least one peptide conjugated to the macromer.

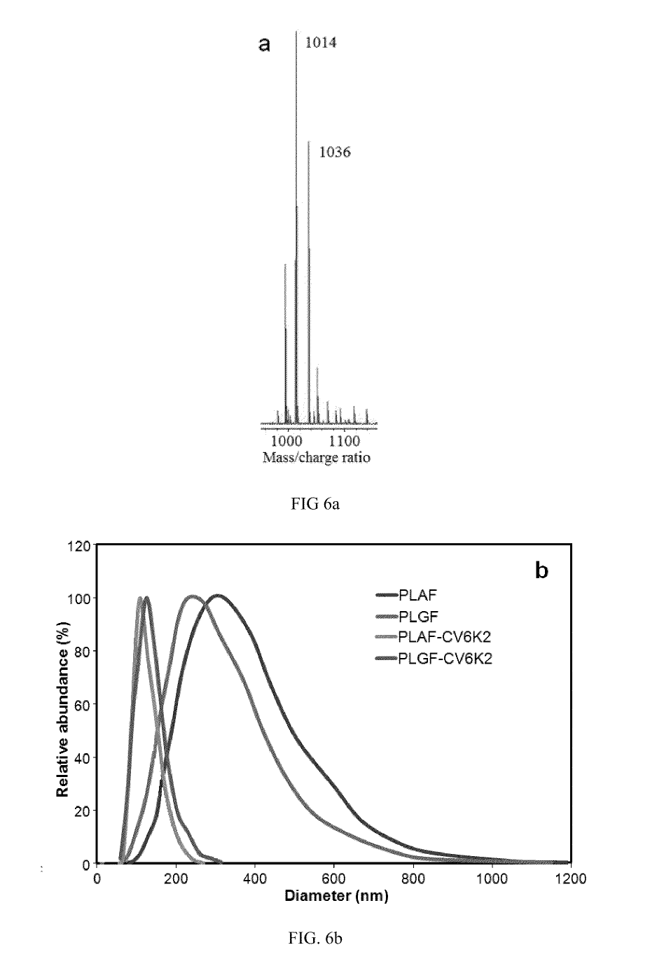

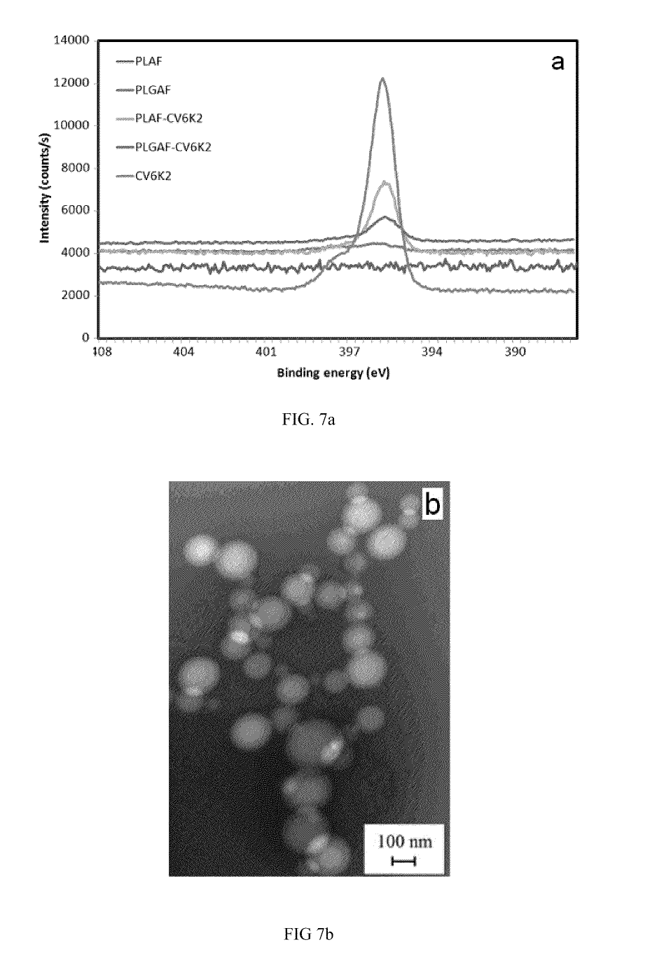

The size and distribution of the NPs is shown in FIG. 6b. DLS results show an average diameter of 70 nm for particles made only with CV6K2 peptide, with a very narrow size distribution. The addition of this peptide to the PLAF and PLGF macromers reduced the size and distribution of the NPs made only with the macromers. The NP size decreased from 300 to 100 nm for PLAF and decreased from 230 to 120 nm for PLGF NPs. The size distributions also decreased from 60-1200 nm to 50-300 nm for both macromers. This demonstrated that the addition of the peptide helped in the self-assembly process of the macromers into NPs. The NPs were also characterized by XPS to determine the surface characteristics of the NPs. The results are shown in FIG. 7a. The nitrogen peak is at a binding energy value of 396 eV. The CV6K2 and the peptide-conjugated NPs exhibited a peak at this value, whereas the PLAF and PLGF peptides did not. This confirmed the presence of the peptide on the surface of PLAF-CV6K2 and PLGF-CV6K2 NPs.

The NP morphology and structure was observed by electron microscopy, as show in FIG. 7b. TEM images show spherical particles with a narrow distribution. Comparison with size of the corresponding PLAF and PLGF NPs show a reduced size and distribution. The polymer-CV6K2 NPs are contemplated as having a layered structure, with an interior and exterior layers consisting mainly of peptide chains in contact with the aqueous environment, and a hydrophobic middle layer, in which the polymer is concentrated. Since PLAF and PLGF macromers, by themselves, are hydrophobic, 10% wt PLEOF macromers must be added to create nanosized particles. The PLEOF acts as a “surfactant” due to the presence of ethylene oxide units, which makes it less hydrophilic. This creates a solid “core and shell” structure, with the less hydrophobic PLEOF in the outer shell and solid PLGF in the more hydrophobic core.

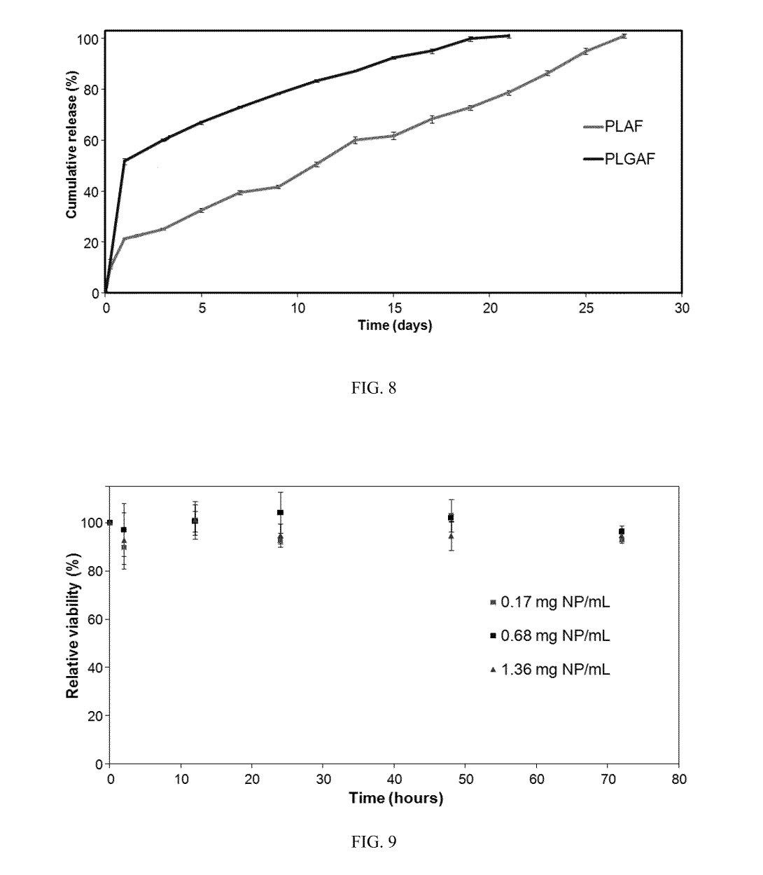

Release of Paclitaxel from the NPs is shown in FIG. 8. The peptide, by itself, did not have the structural integrity of the PLAF-CV6K2 and PLGF-CV6K2, so the release occurred in burst for 5 days. Encapsulation efficiency of the PLAF-CV6K2 and PLGF-CV6K2 NPs was 92 and 88% of the initial amount, respectively. The PLAF NPs had a burst release of 21%, while the PLGF showed a burst release of 52%, both within 24 hours. The release was relatively linear for both macromers, with 27 days for PLAF and 21 days for PLGF. Since PLGF is more hydrophilic, degradation of the matrix and diffusion of the encapsulated species is faster. Paclitaxel is a hydrophobic drug, so it would tend to remain dispersed in the hydrophobic phase than in water, so release would be slower for PLAF NPs when suspended in aqueous solution. Degradation corresponded well to the release profile, with complete degradation in 3 weeks for PLGF-CV6K2 and 4 weeks for PLAF-CV6K2.

Results indicated that the cells could take up the particles after 1 day, with an average uptake of PLAF-CV6K2 NPs of 45%, 88% and 93% uptake for 1×, 4×, and 8× concentrations, respectively, and 44%, 86% and 93% for PLGF-CV6K2 NPs. It was deduced that the NPs were taken up by pinocytosis (for smaller diameter NPs) or phagocytosis (for large NPs). The positively-charged NPs would also be attracted to the negatively-charged cell membrane, facilitating attachment for uptake mechanisms. Cell viability was not affected in 3 days, as shown in FIG. 9. The high uptake of these NPs shows their viability as intracellular carriers.

Peptidomimetic NPs were synthesized and characterized after conjugation of a self assembly peptide to polylactide fumarate NPs. The addition of the peptide to the macromer reduced particle size and distribution of the NPs. Degradation of the NPs was in 3-4 weeks, with an almost linear profile of release of Paclitaxel during this period. The NPs could be taken up by cells and cell viability by incubation with the NPs. These NPs could potentially be used for the delivery of active species in a selective manner.

### Example 3

### In Vivo Effect of the NPs on Host Toxicity and Anti-Tumor Efficacy

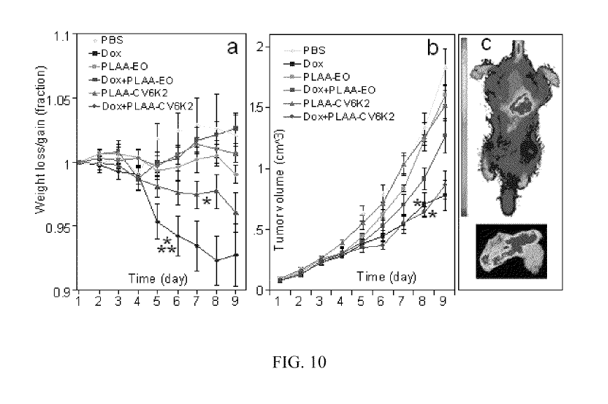

In accordance with the present disclosure, a mouse breast cancer MTCL grown under the back skin of syngeneic C3H mice was utilized. When the tumor size reached 300 mm³, mice were randomly divided into 6 groups (10 mice/group) and received one of these treatments by tail vein injection: PBS, PLAA-EO NPs, PLAA-CV6K2 NPs, Dox, Dox+PLAA-EO NPs, and Dox+PLAA-CV6K2 NPs. The injected Dox amount was 6 mg/kg of body weight. After the treatment, body weight and tumor size were measured daily and the results are shown in FIGS. 10a and 10b, respectively. PBS (yellow), PLAA-EO NPs (light green), and PLAA-CV6K2 (pink) groups had weight gain suggesting NPs alone did not have host toxicity. The Dox (blue) group had the highest host toxicity indicated by the highest body weight loss. Dox+PLAA-CV6K2 (red) treatment had the same anticancer effect as Dox, while it had significantly lower host toxicity as demonstrated by less body weight loss. Compared to Dox treatment, Dox+PLAA-EO NPs had lower toxicity to both the host and tumor. These results suggest that the ionic interaction of PLAA-CV6K2 NPs with the surface of tumor cells resulted in higher tumor toxicity (lower tumor growth rate) with significantly reduced host toxicity. These results point to the importance of cell-NP interaction and penetration of the NPs in the tumor cell to increase tumor toxicity while reducing host toxicity. Near-infrared (NIR) imaging was used to determine in vivo distribution of the NPs loaded with near-infrared dye IRDye 800RS Carboxylate (absorption at 786 nm). The image in pseudo colors 4 h after injection of the NPs in Apc^{Min/+} mice with intestinal tumor is shown in FIG. 10c. The infrared intensity in the intestinal region was at least 100 times higher than the other regions (confirmed by sacrificing the animal and directly imaging the intestinal tissue as shown in the inset of FIG. 10c).

In the interests of brevity and conciseness, any ranges of values set forth in this specification are to be construed as written description support for claims reciting any sub-ranges having endpoints which are whole number values within the specified range in question. By way of a hypothetical illustrative example, a disclosure in this specification of a range of 1-5 shall be considered to support claims to any of the following sub-ranges: 1-4; 1-3; 1-2; 2-5; 2-4; 2-3; 3-5; 3-4; and 4-5.

These and other modifications and variations to the present disclosure can be practiced by those of ordinary skill in the art, without departing from the spirit and scope of the present disclosure, which is more particularly set forth in the appended claims. In addition, it should be understood that aspects of the various embodiments can be interchanged both in whole or in part. Furthermore, those of ordinary skill in the art will appreciate that the foregoing description is by way of example only, and is not intended to limit the disclosure.

What is claimed is:

1. A self-assembling biodegradable nanoparticle comprising:
a degradable synthetic polymer chain, a sequence of non-polar amino acids, and a sequence of ionic amino acids, the nanoparticle having a diameter of from about 50 nm to about 150 nm.

2. The nanoparticle of claim 1, wherein the synthetic polymer chain comprises poly(actide-co-glycolide fumarate), poly(lactide fumarate), polycaprolactone, or combinations thereof.

3. The nanoparticle of claim 1, wherein the sequence of non-polar amino acids comprises valine, glycine, alanine, leucine, isoleucine, methionine, proline, phenylalanine, tryptophan, or combinations thereof.

4. The nanoparticle of claim 1, wherein the sequence of ionic amino acids comprises lysine, arginine, histidine, aspartic acid, glutamic acid, or combinations thereof.

5. The nanoparticle of claim 1, further comprising a chemotherapeutic agent.

6. The nanoparticle of claim 5, wherein the chemotherapeutic agent comprises Paclitaxel.

7. The nanoparticle of claim 1, wherein the nanoparticle has a diameter of from about 75 nm to about 125 nm.

8. The nanoparticle of claim 1, wherein the nanoparticle has a bi-layer structure.

9. The nanoparticle of claim 1, wherein the nanoparticle comprises peptide chains on an outer layer and polymer chains on an inner layer.

10. The nanoparticle of claim 1, wherein the inner layer is hydrophobic.

11. The nanoparticle of claim 1, wherein the nanoparticle is degradable in less than 5 weeks.

12. The nanoparticle of claim 1, wherein the nanoparticle is degradable in less than 4 weeks.

13. The nanoparticle of claim 1, wherein the nanoparticle is degradable in less than 3 weeks.

14. A self-assembling biodegradable nanoparticle comprising:
Cys-Val-Val-Val-Val-Val-Val-Lys-Lys conjugated with a synthetic polymer, the nanoparticle having a diameter of from about 50 nm to about 150 nm; and
a therapeutic agent, the nanoparticle configured to have a generally linear profile of release for the therapeutic agent.

15. The nanoparticle of claim 14, wherein the synthetic polymer comprises poly(actide-co-glycolide fumarate).

16. The nanoparticle of claim 14, wherein the synthetic polymer comprises poly(lactide fumarate).

17. The nanoparticle of claim 14, wherein the synthetic polymer comprises polycaprolactone.

18. The nanoparticle of claim 14, wherein the therapeutic agent comprises a chemotherapeutic agent.

19. The nanoparticle of claim 18, wherein the chemotherapeutic agent comprises Paclitaxel.

20. The nanoparticle of claim 14, wherein the nanoparticle is degradable in less than 5 weeks.
